(19) United States
(12) Patent Application Publication
(10) Pub. No.: US 2010/0120913 A1
(43) Pub. Date: May 13, 2010

(54) RESIN CATALYZED AND STABILIZED PERACID COMPOSITIONS AND ASSOCIATED METHODS

(76) Inventors: Brian G. Larson, Orem, UT (US); Daryl J. Tichy, Orem, UT (US)

Correspondence Address: THORPE NORTH & WESTERN, LLP., P.O. Box 1219, SANDY, UT, 84091-1219, US

(21) Appl. No.: 12/617,355

(22) Filed: Nov. 12, 2009

Related U.S. Application Data
(60) Provisional application No. 61/113,819, filed on Nov. 12, 2008.
(60) Provisional application No. 61/119,995, filed on Dec. 4, 2008.

(51) Int. Cl. A01N 37/00; C07C 409/26; A01P 1/00

(52) U.S. Cl. 514/557; 562/3

(57) ABSTRACT

Methods, compositions, and system for making and stabilizing peracids in peracid-containing compositions are provided. The method of making can include the contacting of a cationic exchange resin with a carboxylic acid and hydrogen peroxide containing solution and maintaining the peracid in contact with the solution for a period of at least 24 hours to form a peracid. The solution to cationic exchange resin weight ratio used in the method is at least 10:1 w/w. The method of stabilizing can similarly include the maintaining the contact between the peracid-containing composition in contact with the cationic exchange resin during the storage of the peracid-containing composition. The methods and systems are particularly useful for peracid-containing disinfectant compositions.

This application claims the benefit of U.S. Provisional Patent Application Nos. 61/113,819, filed Nov. 12, 2008, and 61/119,995, filed Dec. 4, 2008.

### BACKGROUND

Peracid and peracid-containing compositions are used in a wide variety of applications including oxidants, stain removers, and microbicides, amongst others. Unfortunately, the advantageous high reactivity possessed by the peracids also makes them difficult to store for extended periods of time. Specifically, peracids tend to react or degrade fairly rapidly thereby lessening their long-term effectiveness and significantly reducing their shelf life. As such, research continues into methods of stabilizing peracid compositions or peracid concentrations in solution.

### DETAILED DESCRIPTION

Reference will now be made to the exemplary embodiments, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It is also to be understood that the terminology used herein is used for the purpose of describing particular embodiments only. The terms are not intended to be limiting unless specified as such.

It must be noted that, as used in this specification and the appended claims, the singular forms “a,” “an,” and “the” include plural referents unless the content clearly dictates otherwise.

The term “cationic exchange resin” refers to an insoluble matrix (or structure) often, but necessarily, in the form of small (1-2 mm diameter) beads, usually white or yellowish, fabricated from an organic polymer substrate. The material can have a highly developed structure of pores on its surface which are capable of easily trapping and releasing ions.

The term “equilibrium,” when used to with respect to the peracid component of the compositions and solutions of the present application, refers to a state in which the rate of formation of the peracid is approximately equal to the rate of degradation or breakdown of the peracid. Thus, the composition can be said to be at equilibrium, and can further be said to be equilibrating as peracid is replenished by the presence of cationic exchange resin, for example. As used herein, the term equilibrium does not require that the formation and degradation rates be exactly equal, rather that the rates be approximately equal, namely within 5% of each other, preferably within 3%.

The term “colloidal transition metals” refers to colloidal particles of elemental transitional metals or the alloys of such elemental transition metals. Colloidal transition metals are distinct from salts and oxides of transition metals. Accordingly, compounds such as silver oxide, silver nitrate, silver chloride, silver bromide, silver iodide, and the like are not colloidal transition metals under the present definition.

The term “stable,” “stabilize,” “stabilized,” “stabilizer,” or the like can refer to peracids that are stabilized in the presence of a cationic exchange resin, or where peracid is replenished as a result of contact with the cationic exchange resin. Thus, the peracid can be essentially maintained in equilibrium or can be an equilibrating composition as a result of contact with cationic exchange resin in a batch composition over a period of time, e.g., several hours to several months or even years. Thus, it is noted that stabilization of a composition can include stabilization of a concentration of the composition and still be considered stable.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of about 1 wt % to about 20 wt % should be interpreted to include not only the explicitly recited limits of about 1 wt % and about 20 wt %, but also to include individual weights such as 2 wt %, 11 wt %, 14 wt %, and sub-ranges such as 10 wt % to 20 wt %, 5 wt % to 15 wt %, etc.

In accordance with these definitions and embodiments of the present invention, a discussion of the various systems and methods is provided including details associated therewith. This being said, it should be noted that various embodiments will be discussed as they relate to the systems and methods. Regardless of the context of the specific details as they are discussed for any one of these embodiments, it is understood that such discussion relates to other all other embodiments as well.

Accordingly, the present disclosure provides for methods and associated systems for stabilizing peracids or peracid concentrations in peracid-containing compositions. In particular, in one embodiment, a method of stabilizing a peracid or peracid concentration in a peracid-containing composition during storage is provided. The method includes the steps of providing a peracid-containing composition and a cationic exchange resin, and then stabilizing the peracid-containing composition or peracid concentration by contacting the peracid-containing composition with the cationic exchange resin during storage.

In another embodiment, a system for stabilizing a peracid or peracid concentration during storage is provided. The system can include a peracid-containing composition, a container configured for retaining the peracid-containing composition, and a cationic exchange resin. The system can be configured such that the peracid-containing composition can be disposed within the container. The cationic exchange resin can be disposed in the system so as to be in contact with the peracid-containing composition during a storage period of at least 1 hour.

In another embodiment, a method of making a peracid is provided. The method includes admixing a carboxylic acid with a peroxide and water to form a solution. The solution is contacted with a cationic exchange resin and the cationic exchange resin is maintained in contact with the solution for a period of time of at least 24 hours thereby forming a peracid. The weight ratio of the solution to the cationic exchange resin is at least 10:1 (w/w).

In yet another embodiment, an equilibrating peracid-containing composition is provided. The equilibrating peracid-containing composition comprises a solution and a cationic exchange resin. The solution includes water, a peracid, and a peroxide. The composition is in equilibrium with respect to the peracid and the weight ratio of the solution to the cationic exchange resin is at least 10:1 (w/w). In one embodiment, the cationic exchange resin can facilitate replenishment of the peracid in order to maintain the equilibrium of the solution with respect to the peracid.

It is noted that when discussing peracid-containing composition, systems for stabilizing peracid or peracid concentration during storage, or a methods of making or stabilizing a peracid-containing composition or peracid concentration, each of these discussions can be considered applicable to each of these embodiments, whether or not they are explicitly discussed in the context of that embodiment. Thus, for example, in discussing the cationic exchange resins used in a particular peracid stabilizing system, those cationic exchange resins can also be used in a method for stabilizing peracids or peracid concentrations, and vice versa.

With the above-described embodiments in mind, the compositions, systems, and methods of the present invention can aid in making and stabilizing a large variety of peracids. Examples of peracids which can be stabilized by the systems and methods of the present invention include, but are not limited to peracetic acid, percitric acid, performic acid, peroxalic acid, perpropanoic acid, perlactic acid, perbutanoic acid, perpentanoic acid, perhexanoic acid, peradipic acid, perbenzoic acid, permalic acid, permalonic acid, persuccinic acid, perglutaric acid, peradipic acid, permaleic acid, perfumaric acid, and mixtures thereof. In one embodiment, the peracid being stabilized is peracetic acid. In another embodiment, the peracid is percitric acid.

The peracids made and/or stabilized by the methods and systems of the present application can be produced or manufactured by any method known in the art. In one embodiment, the peracid being stabilized can be produced by adding or contacting an amount of cationic exchange resin to a solution of water, a carboxylic acid, and hydrogen peroxide. In one embodiment, solution and the cationic exchange resin are maintained in contact for a period of at least 24 hours. In another embodiment, the solution and cationic exchange resin can be maintained in contact for a period of at least 5 days. In yet another embodiment, the solution and cationic exchange resin can be maintained in contact for a period of time of at least 10 days. In yet a further embodiment, the solution and cationic exchange resin can be maintained in contact for a period of at least 15 days.

Generally, any carboxylic acid can be used in the manufacturing methods of the present application. Non-limiting examples include acetic acid, citric acid, formic acid, oxalic acid, propanoic acid, lactic acid, butanoic acid, pentanoic acid, hexanoic acid, adipic acid, benzoic acid, malic, malonic, succinic, glutaric, maleic, and mixtures thereof.

The cationic exchange resins used in the systems and methods of the present application can be of any type known in the art, including sulfonated polystyrene resins such as sulfonated polystyrene-divinylbenzene copolymers. Non-limiting examples of commercially available cationic exchange resins include Rohm and Haas Amberlite IR-120 and Amberlite IRA-93, Dow's Dowex-50, Zeocarb 225, Amberlite IR-118, Amberlyst 15, and other related cationic exchange resins, particularly acid exchange resins. The cationic exchange resins can be present in the compositions, systems, and methods of peracid stabilization and production at weight ratios of solution (including at least water, carboxylic acid, and peroxide) to cationic exchange resin of at least 10:1. In one embodiment, the weight ratio of the solution to cationic exchange resin can be at least 25:1. In another embodiment, the weight ratio of the solution to cationic exchange resin can be at least 50:1. In yet another embodiment, the weight ratio of the solution to cationic exchange resin can be at least 100:1.

The amount of cationic resin needed to adequately stabilize the peracids in the peracid-containing composition can vary depending on the concentration of the peracid and the type of peracid concentration being stabilized. In one embodiment, the cationic resin is present at a weight ratio of peracid to cationic exchange resin of about 1:100 w/w to 100,000:1 w/w. In another embodiment, the cationic resin is present at a weight ratio of peracid to cationic exchange resin of about 1:10 w/w to 10000:1 w/w. In one embodiment, the weight ratio of peracid to cationic exchange resin is 100:1 w/w to 100:5 w/w.

The form of the cationic exchange resin used in the present application can vary depending on the nature of the peracid-containing composition, the type of container in which the peracid-containing composition is being stored, the duration of storage, or other variables. In one embodiment, the cationic exchange resin can be present as a small bead or sphere. When the cationic exchange resin beads are used, they can be submersed in the peracid-containing composition. In one embodiment, the cationic exchange resin can be retained and maintained within a compartment present in the container in which the peracid-composition is stored. The compartment can be an integrated element of the storage container or it can be a separate compartment. Regardless of configuration or compartment type, the compartment is typically permeable to the peracid-containing composition in order to allow direct contact between the cationic exchange resin and the peracid in the peracid-containing composition. Examples of compartments that could be used include, but are not limited to, pouches or envelopes made of materials capable of retaining the cationic exchange resin while still being permeable to the peracid-containing composition. Example materials include but are not limited to fine mesh e.g. nylon, porous paper or silk e.g. a tea bag, screens, or other materials known in the art.

In another embodiment, the cationic exchange resin can be present as linear strands of the resin. Single or multiple strands of the cationic exchange resin can be placed into contact with the peracid-containing composition in order enhance the stability. The strands of the cationic exchange resin can be directly submersed into the peracid-containing composition or can be incorporated into the structure of the container in which the peracid-containing composition is stored, e.g. hung from the lid or integrated into the walls of the container.

In addition to the beads and strands, the cationic exchange resin can be integrated directly into the container in which the peracid-containing composition is stored. When the cationic exchange resin is integrated into the container, it is integrated so as to allow continuous contact between the cationic exchange resin and the peracid. For example, in one aspect of the invention, the cationic exchange resin can be coated onto an interior wall of the container. In another aspect, the cationic exchange resin can be integrated into the cap of the container either as a coated layer or as a protrusion that can be in continuous contact with the peracid-containing composition present in the container.

Regardless of the form of the cationic exchange resin, it is desirable that the resin be maintained in contact with the peracid-containing composition during its storage. Accordingly, the step of maintaining the peracid-containing composition in contact with the cationic exchange resin ends when the peracid composition is removed from the container. When the peracid-containing composition is dispensed or removed from the container it can be desirable to be able to mechanically or physically separate the cationic exchange resin, regardless of form, from the dispensed peracid-containing composition. Where necessary, mechanical separation means can be done by filtration or any other means known in the art.

The methods and systems of the present application provide increased storage stability for the peracid-containing compositions. Accordingly, the present invention allows for the storage of peracid-containing compositions for periods of time of 3 days to 365 days. In one embodiment, the storage of the peracid is at least 5 days.

The methods and systems of the present application are generally capable of increasing the stability of a peracid in a peracid-containing composition. Generally, all increases in stability of the peracid in the peracid containing composition are considered to be within the scope of the present application. Specifically, in one embodiment, the stability of the peracid in the peracid-containing composition can be increased by at least about 5% after 1 year when compared to a 1 year storage time of the peracid-containing composition without the cationic exchange resin. In another embodiment, the stability of the peracid in the peracid-containing composition is increased by at least about 50% after 1 year when compared to a 1 year storage time of the peracid-containing composition without the cationic exchange resin.

Although the methods and systems of the present application can be used to stabilize any peracid-containing composition or peracid concentration, the methods can be of particular use for peracid-containing disinfectant compositions. In one embodiment, the peracid-containing disinfectant composition can include a transition metal. Non-limiting examples of transition metals which can be used include ruthenium, rhodium, osmium, iridium, palladium, platinum, copper, gold, silver, alloys thereof, and mixtures thereof. In one embodiment, the transition metal is silver. The metals can be present in either colloidal (i.e. elemental metal or metal alloy) or ionic form (e.g. metal salts), or mixtures thereof. In one embodiment the transition metal can be a colloidal transition metal.

The disinfectant peracid containing composition can also include an alcohol. Generally, any type of alcohol can be included. Non-limiting examples of alcohols which can be included in the disinfectant peracid-containing compositions include aliphatic alcohols and other carbon-containing alcohols, having from 1 to 24 carbons (C₁-C₂₄ alcohol). It is to be noted that “C₁-C₂₄ alcohol” does not necessarily imply only straight chain saturated aliphatic alcohols, as other carbon-containing alcohols can also be used within this definition, including branched aliphatic alcohols, alicyclic alcohols, aromatic alcohols, unsaturated alcohols, as well as substituted aliphatic, alicyclic, aromatic, and unsaturated alcohols, etc. In one embodiment, the aliphatic alcohols can be Cᵢ to C₅ alcohols including methanol, ethanol, propanol and isopropanol, butanols, and pentanols, due to their availability and lower boiling points. In some embodiments it can be advantageous to use a polyhydric alcohol or mixture of polyhydric alcohols as they can be effective in enhancing the disinfectant and sterilant potency of the peracid-containing compositions made by the methods of the present application.

Further it is believed that such polyhydric alcohols can also enhance stability of the peracid-containing compositions. Without being limited by theory, it is believed that the increased number of hydroxyl groups in the polyhydric alcohols enhance the potency of the disinfectant and sterilant solutions by interacting with the aqueous medium and the peracid thereby stabilizing the solution. The increase in the hydroxyl groups may also increase the number of hydroxyl radicals or groups in the disinfectant/sterilant solutions thereby further enhancing the potency or kill ability of the solutions/dispersions. Examples of polyhydric alcohols which can be used in the present application include but are not limited to ethylene glycol (ethane-1,2-diol) glycerin (or glycerol, propane-1,2,3-triol), and propane-1,2-diol. Other non-aliphatic alcohols may also be used including but not limited to phenols and substituted phenols, erucyl alcohol, ricinolyl alcohol, arachidyl alcohol, capryl alcohol, capric alcohol, behenyl alcohol, lauryl alcohol (1-dodecanol), myristyl alcohol (1-tetradecanol), cetyl (or palmityl) alcohol (1-hexadecanol), stearyl alcohol (1-octadecanol), isostearyl alcohol, oleyl alcohol (cis-9-octadecen-1-ol), palmitoleyl alcohol, linoleyl alcohol (9Z, 12Z-octadecadien-1-ol), elaidyl alcohol (9E-octadecen-1-ol), elaidolinoleyl alcohol (9E, 12E-octadecadien-1-ol), linolenyl alcohol (9Z, 12Z, 15Z-octadecatrien-1-ol), elaidolinolenyl alcohol (9E, 12E, 15-E-octadecatrien-1-ol), combinations thereof and the like. Of course, if the disinfectant composition is intended for application to a skin or mucosal surface, it is beneficial to select an alcohol which is safe for these types of applications.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Stabilization of Percitric Acid in a Solution

A percitric acid containing solution was prepared as set out in Table 1.

TABLE 1

| Component | Amount |
|---|---|
| Water (distilled, deionized, 9 megohm) | 744.2 g |
| Percitric Acid | 12.0 g |
| Citric Acid | 84.0 g |
| Hydrogen Peroxide | 84.0 g |
| Disodium Ethylenediaminetetracetic | 0.50 g |
| Acid (EDTA) | |

The solution was divided into two equally proportioned amounts, Solution 1 and Solution 2. A one-gram sample of Amberlite IR-120 was added to each of Solution 1 and Solution 2 as a stabilizer. These solutions were labeled Solution 1 and Solution 2. To Solution 2 a 0.462 g (1000 ppm) amount of dipicolinic acid was also added as a stabilizer. The concentrations of hydrogen peroxide and percitric acid were tested by titration after 11 days and after 112 days. The results are shown in Table 2.

TABLE 2

| | |
|---|---|
| 11 Days | 112 Days |

| | Hydrogen | Percitric | Hydrogen | Percitric |
|---|---|---|---|---|
| | Peroxide | Acid | Peroxide | Acid |
| Solution 1 | 7.38 wt % | 1.09 wt % | 6.61 wt % | 1.49 wt % |
| Solution 2 | 7.31 wt % | 1.22 wt % | — | — |

### Example 2

### Catalyzed Manufacture of Percitric Acid

A percitric acid containing solution was manufactured by using the components set forth in Table 3.

TABLE 3

| Component | Amount |
|---|---|
| Anhydrous Citric Acid | 213.0 g |
| 50% hydrogen peroxide | 309.02 g |
| Amberlite IR-120 | 5.33 g |

The anhydrous citric acid and the 50% hydrogen peroxide were mixed at 30° C. for 30 minutes (until all citric acid was dissolved). The Amberlite IR-120 was added to the resulting solution and the mixture was allowed to react at room temperature for a period of 18 days. After the 18-day period, a sample was removed and titrated in order to determine relative concentrations of the hydrogen peroxide and percitric acid. Hydrogen peroxide was determined to be 18.37 wt % of the solution and percitric acid was determined to be 17.08 wt % of the solution. The maintenance of the Amberlite IR-120 resin in the solution can provide enhanced stabilization to the percitric acid present in the solution (see Example 1).

### Example 3

### Catalyzed Manufacture of Perlactic Acid

A perlactic acid containing solution was manufactured by using the components set forth in Table 4.

TABLE 4

| Component | Amount |
|---|---|
| Lactic Acid | 10.01 g |
| 50% hydrogen peroxide | 15.0 g |
| Amberlite IR-120 | 0.25 g |

The lactic and the 50% hydrogen peroxide were mixed until all lactic acid was dissolved. The Amberlite IR-120 was added to the resulting solution and the mixture was allowed to react at room temperature for a period of 18 days. After the 18-day period, a sample was removed and titrated in order to determine relative concentrations of the hydrogen peroxide and perlactic. Hydrogen peroxide was determined to be 18.61 wt % of the solution and perlactic acid was determined to be 16.32 wt % of the solution. The maintenance of the Amberlite IR-120 resin in the solution can provide enhanced stabilization to the perlactic acid present in the solution (see Example 1).

### Example 4

### Catalyzed Manufacture of Permalic Acid

A permalic acid containing solution was manufactured by using the components set forth in Table 5.

TABLE 5

| Component | Amount |
|---|---|
| Malic Acid | 10.00 g |
| 50% hydrogen peroxide | 15.0 g |
| Amberlite IR-120 | 0.25 g |

The malic acid and the 50% hydrogen peroxide were mixed until all malic acid was dissolved. The Amberlite IR-120 was added to the resulting solution and the mixture was allowed to react at room temperature for a period of 18 days. After the 18-day period, a sample was removed and titrated in order to determine relative concentrations of the hydrogen peroxide and permalic acid. Hydrogen peroxide was determined to be 18.84 wt % of the solution and permalic acid was determined to be 15.93 wt % of the solution. The maintenance of the Amberlite IR-120 resin in the solution can provide enhanced stabilization to the permalic acid present in the solution (see Example 1).

### Example 5

### Catalyzed Manufacture of Permalonic Acid

A permalonic acid containing solution was manufactured by using the components set forth in Table 6.

TABLE 6

| Component | Amount |
|---|---|
| Malonic Acid | 10.00 g |
| 50% hydrogen peroxide | 15.0 g |
| Amberlite IR-120 | 0.25 g |

The malonic acid and the 50% hydrogen peroxide were mixed until all malonic acid was dissolved. The Amberlite IR-120 was added to the resulting solution and the mixture was allowed to react at room temperature for a period of 18 days. After the 18-day period, a sample was removed and titrated in order to determine relative concentrations of the hydrogen peroxide and permalonic acid. Hydrogen peroxide was determined to be 18.84 wt % of the solution and permalonic acid was determined to be 15.93 wt % of the solution. The maintenance of the Amberlite IR-120 resin in the solution can provide enhanced stabilization to the permalonic acid present in the solution (see Example 1).

### Example 6

### Catalyzed Manufacture of Persuccinic Acid

A persuccinic acid containing solution was manufactured by using the components set forth in Table 7.

TABLE 7

| Component | Amount |
|---|---|
| Succinic Acid | 2.00 g |
| 50% hydrogen peroxide | 34.79 g |
| Amberlite IR-120 | 0.050 g |

The succinic acid and the 50% hydrogen peroxide were mixed until all succinic acid was dissolved. The Amberlite IR-120 was added to the resulting solution and the mixture was allowed to react at room temperature for a period of 18 days. After the 18-day period, a sample was removed and titrated in order to determine relative concentrations of the hydrogen peroxide and permalonic acid. Hydrogen peroxide was determined to be 29.1 wt % of the solution and persuccinic acid was determined to be 1.94 wt % of the solution. The maintenance of the Amberlite IR-120 resin in the solution can provide enhanced stabilization to the persuccinic acid present in the solution (see Example 1).

### Example 7

### Catalyzed Manufacture of Percitric Acid

A percitric acid containing solution is manufactured by using the components set forth in Table 8.

TABLE 8

| Component | Amount |
|---|---|
| Anhydrous Citric Acid | 213.0 g |
| 50% hydrogen peroxide | 309.02 g |
| Amberlite IR-120 | 52.0 g |

The anhydrous citric acid and the 50% hydrogen peroxide are mixed at 30° C. for 30 minutes (until all citric acid is dissolved). The Amberlite IR-120 is added to the resulting solution and the mixture is allowed to react at room temperature for a period of 18 days. The maintenance of the Amberlite IR-120 resin in the solution can provide enhanced stabilization to the percitric acid present in the solution (see Example 1).

### Example 8

### Catalyzed Manufacture of Percitric Acid

A percitric acid containing solution is manufactured by using the components set forth in Table 9.

TABLE 9

| Component | Amount |
|---|---|
| Anhydrous Citric Acid | 1065.0 g |
| 50% hydrogen peroxide | 1545.1 g |
| Amberlite IR-120 | 103.0 g |

The anhydrous citric acid and the 50% hydrogen peroxide are mixed at 30° C. for 30 minutes (until all citric acid is dissolved). The Amberlite IR-120 is added to the resulting solution and the mixture is allowed to react at room temperature for a period of 18 days. The maintenance of the Amberlite IR-120 resin in the solution can provide enhanced stabilization to the percitric acid present in the solution (see Example 1).

### Example 9

### Catalyzed Manufacture of Percitric Acid

A percitric acid containing solution is manufactured by using the components set forth in Table 10.

TABLE 10

| Component | Amount |
|---|---|
| Anhydrous Citric Acid | 355.0 g |
| 50% hydrogen peroxide | 515.1 g |
| Amberlite IR-120 | 17.1 g |

The anhydrous citric acid and the 50% hydrogen peroxide are mixed at 30° C. for 30 minutes (until all citric acid is dissolved). The Amberlite IR-120 is added to the resulting solution and the mixture is allowed to react at room temperature for a period of 18 days. The maintenance of the Amberlite IR-120 resin in the solution can provide enhanced stabilization to the percitric acid present in the solution (see Example 1).

### Example 10

### Catalyzed Manufacture of Percitric Acid

A percitric acid containing solution is manufactured by using the components set forth in Table 11.

TABLE 11

| Component | Amount |
|---|---|
| Anhydrous Citric Acid | 2485.0 g |
| 50% hydrogen peroxide | 3605.7 g |
| Amberlite IR-120 | 60.7 g |

The anhydrous citric acid and the 50% hydrogen peroxide are mixed at 30° C. for 30 minutes (until all citric acid is dissolved). The Amberlite IR-120 is added to the resulting solution and the mixture is allowed to react at room temperature for a period of 18 days. The maintenance of the Amberlite IR-120 resin in the solution can provide enhanced stabilization to the percitric acid present in the solution (see Example 1).

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method of stabilizing a peracid in a peracid-containing composition during storage, comprising:
providing a peracid-containing composition and a cationic exchange resin, and
stabilizing the peracid-containing composition by contacting the peracid-containing composition with the cationic exchange resin during storage.

2. A method as in claim 1, wherein the cationic exchange resin is a sulfonated polystyrene.

3. A method as in claim 2, wherein the cationic exchange resin is a sulfonated polystyrene-divinylbenzene copolymer.

4. A method as in claim 1, wherein the peracid is selected from the group consisting of peracetic acid, percitric acid, performic acid, peroxalic acid, perpropanoic acid, perlactic acid, perbutanoic acid, perpentanoic acid, perhexanoic acid, peradipic acid, perbenzoic acid, permalic acid, permalonic, persuccinic, perglutaric, peradipic, permaleic, perfumaric and mixtures thereof.

5. A method as in claim 4, wherein the peracid is peracetic acid.

6. A method as in claim 4, wherein the peracid is percitric acid.

7. A method as in claim 1, wherein the cationic exchange resin is a bead or a plurality of beads of cationic exchange resin.

8. A method as in claim 7, wherein the bead or plurality of beads is submersed in the peracid-containing composition.

9. A method as in claim 1, wherein the cationic exchange resin is a strand or plurality of strands of the cationic exchange resin.

10. A method as in claim 9, wherein the strand or plurality of strands are submersed in the peracid-containing composition.

11. A method as in claim 1, wherein the peracid-containing composition is present in a container.

12. A method as in claim 11, wherein the cationic exchange resin is integrated with the container so that it is in continuous contact with the peracid during storage.

13. A method as in claim 12, wherein the cationic exchange resin is coated onto an interior wall of the container.

14. A method as in claim 11, wherein the cationic exchange resin is maintained within a compartment, said compartment being permeable to the peracid-containing composition during storage.

15. A method as in claim 11, wherein the container has a cap and said cationic exchange resin is integrated with the cap.

16. A method as in claim 11, wherein the step of maintaining the peracid-containing composition in contact with the cationic exchange resin ends when the peracid composition is removed from the container.

17. A method as in claim 1, wherein the storage of the peracid-containing composition is for a time period of 5 days to 365 days.

18. A method as in claim 1, wherein the storage of the peracid-containing composition is for a period of time of at least 5 days.

19. A method as in claim 1, wherein the cationic exchange resin is present in a form that is mechanically separable from the peracid-containing composition.

20. A method as in claim 1, wherein the cationic resin is present at a weight ratio of peracid to cationic exchange resin of about 1:100 w/w to 100,000:1 w/w.

21. A method as in claim 1, wherein the method increases the stability of the peracid in the peracid-containing composition by at least about 5% after 1 year when compared to a 1 year storage time of the peracid-containing composition without the cationic exchange resin.

22. A method as in claim 1, wherein the method increases the stability of the peracid in the peracid-containing composition by at least about 50% after 1 year when compared to a 1 year storage time of the peracid-containing composition without the cationic exchange resin.

23. A method as in claim 1, wherein the peracid-containing composition is a disinfectant composition.

24. A method as in claim 23, wherein the peracid-containing composition includes a transition metal.

25. A method as in claim in claim 24, wherein the transition metal is a colloidal transition metal.

26. A method as in claim 24, wherein the transition metal is selected from the group consisting of ruthenium, rhodium, osmium, iridium, palladium, platinum, copper, gold, silver, alloys thereof, and mixtures thereof.

27. A method as in claim 24, wherein the transition metal is silver.

28. A method as in claim 23, wherein the peracid-containing composition includes an alcohol.

29. A method as in claim 28, wherein the alcohol is a polyhydric alcohol.

30. A method as in claim 1, wherein the peracid in the peracid-containing composition is manufactured by adding the cationic exchange resin to a mixture of a carboxylic acid and hydrogen peroxide.

31. A method as in claim 30, wherein the carboxylic acid is selected from the group consisting of acetic acid, citric acid, formic acid, oxalic acid, propanoic acid, lactic acid, malic acid, malonic acid, succinic acid, butanoic acid, pentanoic acid, hexanoic acid, adipic acid, benzoic acid, and mixtures thereof.

32. A method as in claim 1, wherein the peracid in the peracid-containing composition is manufactured by adding the cationic exchange resin to a mixture of a carboxylic acid and hydrogen peroxide.

33. A method as in claim 32, wherein the carboxylic acid is selected from the group consisting of acetic acid, citric acid, formic acid, oxalic acid, propanoic acid, lactic acid, malic acid, malonic acid, succinic acid, butanoic acid, pentanoic acid, hexanoic acid, adipic acid, benzoic acid, and mixtures thereof.

34. A system for stabilizing a peracid during storage, comprising:
a peracid-containing composition;
a container configured for retaining the peracid-containing composition; and
a cationic exchange resin;
wherein the peracid-containing composition is disposed within the container and the cationic exchange resin is disposed in the system so as to be in contact with the peracid-containing composition during a storage period of at least 3 days.

35. A system as in claim 34, wherein the cationic exchange resin is a sulfonated polystyrene.

36. A system as in claim 34, wherein the cationic exchange resin is a sulfonated polystyrene-divinylbenzene copolymer.

37. A system as in claim 34, wherein the peracid is selected from the group consisting of peracetic acid, percitric acid, performic acid, peroxalic acid, perpropanoic acid, perlactic acid, perbutanoic acid, perpentanoic acid, perhexanoic acid, peradipic acid, permalonic acid, permalic acid, persuccinic acid, perbenzoic acid, and mixtures thereof.

38. A system as in claim 37, wherein the peracid is peracetic acid.

39. A system as in claim 37, wherein the peracid is percitric acid.

40. A system as in claim 34, wherein the cationic exchange resin is present as a small bead.

41. A system as in claim 40, wherein the cationic exchange resin beads are submersed in the peracid-containing composition.

42. A system as in claim 34, wherein the cationic exchange resin is present as strands of the cationic exchange resin.

43. A system as in claim 42, wherein the strands are submersed in the peracid-containing composition.

44. A system as in claim 34, wherein the cationic exchange resin is integrated into the container so that it is in continuous contact with the peracid during storage.

45. A system as in claim 34, wherein the cationic exchange resin is coated onto an interior wall of the container.

46. A system as in claim 34, wherein the cationic exchange resin is maintained within a compartment, said compartment being permeable to the peracid-containing composition.

47. A system as in claim 34, wherein the storage period is at least 5 days to 365 days.

48. A system as in claim 34, wherein the container is configured to retain the peracid-containing composition for a period of time of at least 5 days.

49. A system as in claim 34, wherein the cationic resin is present in a form that can be mechanically separated from the peracid-containing composition.

50. A system as in claim 34, wherein the cationic resin is present at a weight ratio of peracid to cationic resin of about 1:100 w/w to 100,000:1 w/w.

51. A system as in claim 34, wherein the system increases the stability of the peracid in the peracid-containing composition by at least about 5% after 1 year when compared to a 1 year storage time of the peracid-containing composition in a system without the cationic exchange resin.

52. A system as in claim 34, wherein the system increases the stability of the peracid in the peracid-containing composition by at least about 50% after 1 year when compared to a 1 year storage time of the peracid-containing composition without the cationic exchange resin.

53. A system as in claim 34, wherein the peracid-containing composition is a disinfectant composition.

54. A system as in claim 53, wherein the peracid-containing composition includes a transition metal.

55. A system as in claim 54, wherein the transition metal is a colloidal transition metal.

56. A system as in claim 54, wherein the transition metal is selected from the group consisting of ruthenium, rhodium, osmium, iridium, palladium, platinum, copper, gold, silver, alloys thereof, and mixtures thereof.

57. A system as in claim 54, wherein the transition metal is silver.

58. A system as in claim 34, wherein the peracid-containing composition includes an alcohol.

59. A system as in claim 58, wherein the alcohol is a polyhydric alcohol.

60. A method of making a peracid, comprising:
admixing a carboxylic acid with a peroxide and water to form a solution;
contacting the solution with a cationic exchange resin; and
maintaining the cationic exchange resin in contact with the solution for a period of time of at least 24 hours in order to form the peracid,
wherein the weight ratio of the solution to the cationic exchange resin is at least 10:1 (w/w).

61. A method as in claim 60, wherein the cationic exchange resin is a sulfonated polystyrene.

62. A method as in claim 61, wherein the cationic exchange resin is a sulfonated polystyrene-divinylbenzene copolymer.

63. A method as in claim 60, wherein the carboxylic acid is selected from the group consisting of acetic acid, citric acid, formic acid, oxalic acid, propanoic acid, lactic acid, butanoic acid, pentanoic acid, hexanoic acid, adipic acid, benzoic acid, malic acid, malonic acid, succinic acid, glutaric acid, adipic acid, maleic acid, fumaric acid, and mixtures thereof.

64. A method as in claim 60, wherein the cationic exchange resin is present as a bead or a plurality of beads of cationic exchange resin.

65. A method as in claim 64, wherein the contacting includes submersing the bead or plurality of beads in the solution.

66. A method as in claim 60, wherein the contacting includes disposing the solution in a container coated with the cationic exchange resin.

67. A method as in claim 60, wherein the period of time is at least 5 days.

68. A method as in claim 60, wherein the period of time is at least 10 days.

69. A method as in claim 60, wherein the period of time is at least 15 days.

70. A method as in claim 60, wherein the weight ratio of the solution to the cationic exchange resin is at least 25:1 (w/w).

71. A method as in claim 60, wherein the weight ratio of the solution to the cationic exchange resin is at least 50:1 (w/w).

72. A method as in claim 60, wherein the weight ratio of the solution to the cationic exchange resin is at least 100:1 (w/w).

73. A method as in claim 60, wherein the solution further includes an alcohol.

74. An equilibrating peracid-containing composition, comprising:
a solution including water, a peracid, and a peroxide; and
a cationic exchange resin;
wherein the composition is in equilibrium with respect to the peracid and wherein the weight ratio of the solution to the cationic exchange resin is at least 10:1 (w/w).

75. A composition as in claim 74, wherein the cationic exchange resin is a sulfonated polystyrene.

76. A composition as in claim 75, wherein the cationic exchange resin is a sulfonated polystyrene-divinylbenzene copolymer.

77. A composition as in claim 74, wherein the peracid is selected from the group consisting of peracetic acid, percitric acid, performic acid, peroxalic acid, perpropanoic acid, perlactic acid, perbutanoic acid, perpentanoic acid, perhexanoic acid, peradipic acid, perbenzoic acid, permalic acid, permalonic, persuccinic, perglutaric, peradipic, permaleic, perfumaric and mixtures thereof.

78. A composition as in claim 74, wherein the cationic exchange resin is present as one or more small beads.

79. A composition as in claim 74, wherein the weight ratio of the solution to the cationic exchange resin is at least 25:1 (w/w).

80. A composition as in claim 74, wherein the weight ratio of the solution to the cationic exchange resin is at least 50:1 (w/w).

81. A composition as in claim 74, wherein the weight ratio of the solution to the cationic exchange resin is at least 100:1 (w/w).

82. A composition as in claim 74, wherein the solution includes an alcohol.

83. A composition as in claim 74, wherein the cationic exchange resin facilitates replenishment of the peracid thereby maintaining the composition in equilibrium.
